(19) United States
(12) Patent Application Publication
(10) Pub. No.: US 2026/0227374 A1
(43) Pub. Date: Aug. 6, 2026

(54) METHOD FOR PROVIDING FLAVOR INFORMATION, INFORMATION PROCESSING APPARATUS, FLAVOR INFORMATION PROVIDING SYSTEM, AND NON-TRANSITORY COMPUTER READABLE MEDIUM

(71) Applicant: SHIMADZU CORPORATION, Kyoto-shi, Kyoto (JP)

(72) Inventor: Keiko MATSUMOTO, Kyoto-shi, Kyoto (JP)

(73) Assignee: SHIMADZU CORPORATION, Kyoto-shi, Kyoto (JP)

(21) Appl. No.: 19/150,497

(22) PCT Filed: Jan. 23, 2024
(86) PCT No.: PCT/JP2024/001816

§ 371 Date: Jul. 23, 2025

(30) Foreign Application Priority Data
Jan. 26, 2023 (JP) 2023-010246

(51) Int. Cl. G01N 33/02; A23L 5/00; G01N 30/88

(52) U.S. Cl. CPC G01N 33/02; A23L 5/00; G01N 30/88

(57) ABSTRACT

A method for providing flavor information related to two or more food products is provided. The method includes a step of acquiring analysis results of respective components of the two or more food products (S10), a step of identifying, using a result of a statistical test that uses the analysis results of the respective components of the two or more food products, a substance that affects a difference in flavor between the two or more food products (S20), and a step of outputting, using the analysis result of the identified substance from among the analysis results of the respective components of the two or more food products, flavor information related to the two or more food products (S30).

### TECHNICAL FIELD

The present disclosure relates to providing flavor information for food products.

### BACKGROUND ART

Conventionally, various proposals have been made regarding the evaluation of the flavor of food products. In the field of food product manufacturing and development, specific evaluators assess the taste of prototypes, and Non-Patent Literature 1 discloses a method for training such evaluators (panels).

### PRIOR ART DOCUMENTS

### Non-Patent Literature

[Non-Patent Literature 1] Hideko Furukawa, “Panel no Kunren to Shohin Kaihatsu” (Training of Panels and Product Development), Journal of the Brewing Society of Japan, Japan, Brewing Society of Japan, Jun. 15, 1983, Vol. 78, No. 6, p. 419-422.

### SUMMARY OF THE INVENTION

### Problem to be Solved by the Invention

However, there is a concern that evaluations relying on human taste lack accuracy. The present disclosure was conceived in view of such circumstances, and an object thereof is to provide a technology for providing information for accurately evaluating the flavor of food products.

### Means for Solving the Problem

A method according to an aspect of the present disclosure is a method for providing flavor information related to two or more food products, the method comprising: a step of acquiring analysis results of respective components of the two or more food products; a step of identifying, using a result of a statistical test that uses the analysis results of the respective components of the two or more food products, a substance that affects a difference in flavor between the two or more food products; and a step of outputting, using the analysis result of the identified substance from among the analysis results of the respective components of the two or more food products, flavor information related to the two or more food products.

An information processing apparatus according to another aspect of the present disclosure comprises: one or more processors; and a storage device storing a program that, when executed by the one or more processors, causes the one or more processors to perform the method described above.

A flavor information providing system according to yet another aspect of the present disclosure comprises: the information processing apparatus described above; and an analysis apparatus that outputs an analysis result of a food product to the information processing apparatus described above.

A program according to still another aspect of the present disclosure, when executed by one or more processors, causes the one or more processors to perform the method described above.

### Effects of the Invention

According to an aspect of the present disclosure, information for accurately evaluating the flavor of food products is provided. In particular, by identifying substances that significantly affect flavor, it is possible to support a quantitative evaluation of flavor.

### BRIEF DESCRIPTION OF THE DRAWINGS

FIG. 1 is a diagram showing a configuration of a flavor information providing system 1.

FIG. 2 is a diagram showing an example of flavor information output by an information processing apparatus 100.

FIG. 3 is a diagram showing an example of flavor information output by the information processing apparatus 100.

FIG. 4 is a diagram showing an example of flavor information output by the information processing apparatus 100.

FIG. 5 is a diagram showing an example of flavor information output by the information processing apparatus 100.

FIG. 6 is a diagram showing an example of flavor information output by the information processing apparatus 100.

FIG. 7 is a diagram showing an example of flavor information output by the information processing apparatus 100.

FIG. 8 is a diagram showing an example of a screen output as an evaluation result for four types of products (Products A-D).

FIG. 9 is a diagram showing an example of a calculation method for five types of indicators.

FIG. 10 is a diagram showing an example of compounds identified as taste-related substances.

FIG. 11 is a diagram showing an example of compounds identified as aroma-related substances.

FIG. 12 is a flowchart of a main routine performed in the information processing apparatus 100.

FIG. 13 is a flowchart of a subroutine for step S20 in FIG. 12.

FIG. 14 is a flowchart of a subroutine for step S30 in FIG. 12, performed for outputting flavor information (FIGS. 2-7).

### DETAILED DESCRIPTION OF THE EMBODIMENTS

Hereinafter, embodiments of the present disclosure will be described in detail with reference to the drawings. In the drawings, the same or corresponding parts are denoted by the same reference numerals, and a description thereof will not be repeated.

### 1. System Configuration

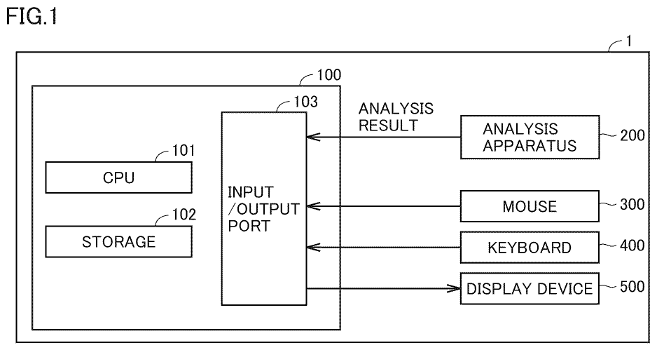

FIG. 1 is a diagram showing a configuration of a flavor information providing system 1. The flavor information providing system 1 mainly includes an information processing apparatus 100 and an analysis apparatus 200. The information processing apparatus 100 acquires an analysis result for a food product from the analysis apparatus 200 and provides information related to the flavor of the food product using the analysis result. The analysis apparatus 200 may be, for example, a liquid chromatograph-mass spectrometer and/or a gas chromatograph-mass spectrometer.

In one implementation, the information processing apparatus 100 is realized by a general-purpose computer. More specifically, the information processing apparatus 100 includes a CPU (Central Processing Unit) 101, a storage 102, and an input/output port 103.

The CPU 101 is configured by one or more processors. The storage 102 is an example of a storage device and non-transitorily stores programs and/or data. The information processing apparatus 100 acquires an analysis result of a food product from the analysis apparatus 200 via the input/output port 103. The information processing apparatus 100 may also be realized by the cooperation of multiple computers. In one implementation, the one or more processors constituting the CPU 101 execute a program non-transitorily stored in the storage 102 (or a storage device outside the information processing apparatus 100 accessible by the one or more processors), whereby the information processing apparatus 100 performs various processes.

A mouse 300, a keyboard 400, and a display device 500 are connected to the information processing apparatus 100. The information processing apparatus 100 accepts external input via the mouse 300 and the keyboard 400, and outputs information by displaying a screen on the display device 500. The information processing apparatus 100 may also include a network interface and may communicate with external information devices via a network.

In the present embodiment, sake is mainly adopted as an example of a food product. However, the food product for which the information processing apparatus 100 provides flavor information is not limited to sake. Any type of product that can be an analysis target by the analysis apparatus 200 can be a subject for information provision.

### 2. Flavor Information

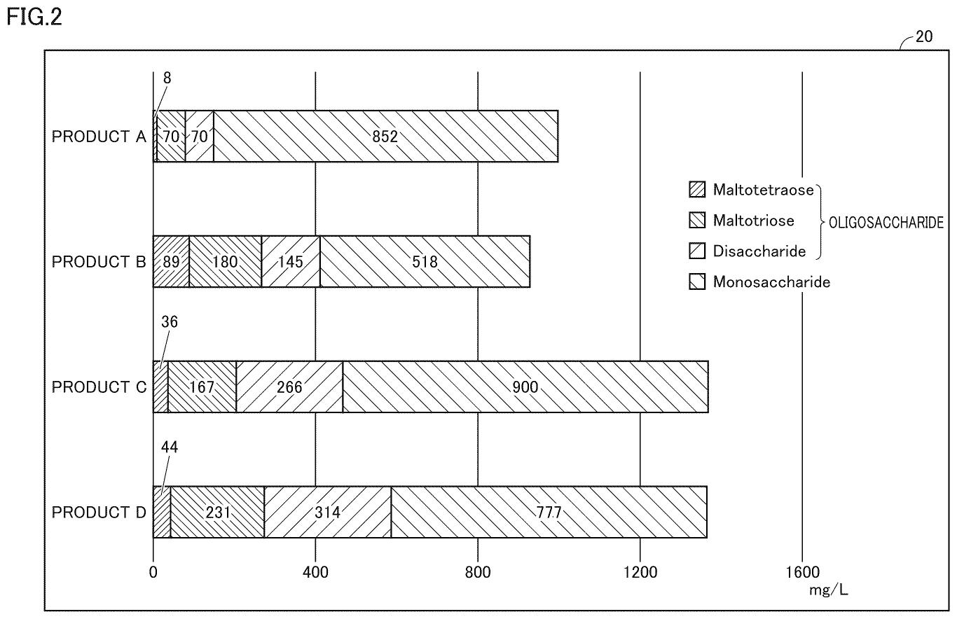

Each of FIGS. 2 to 7 is a diagram showing an example of flavor information output by the information processing apparatus 100.

In one implementation, the information processing apparatus 100 may identify the compounds shown in FIGS. 2 to 7 as substances that affect the difference in flavor among the four types of sake shown in each figure (taste-related substances or aroma-related substances), as will be described later. In one implementation, the mass spectrometer outputs the content of each compound in the food product to the information processing apparatus 100 as an analysis result of the food product. More specifically, the mass spectrometer calculates the content of each compound using the ratio of the peak area of each compound to the peak area of an internal standard substance in a mass spectrogram. The content of each compound may also be calculated using the concentration of the compound in the sample used for analysis and a calibration curve representing the relationship between concentration and content. Further, the content of each compound may be calculated from the peak area value of a mass spectrogram or a chromatogram.

### (Screen 20 of FIG. 2)

The screen 20 of FIG. 2 displays the content of four types of sugars (Monosaccharide, Disaccharide, Maltotriose, Maltotetraose) for each of four types of sake (Products A, B, C, D).

A user can use the information shown on the screen 20 as support for considering the differences in flavor among products A, B, C, and D.

More specifically, as shown on the screen 20, Products C and D have a higher total content of the four types of sugars than Products A and B. Note that Products A and B have a higher rice polishing ratio than Products C and D. Therefore, based on the results shown on the screen 20, the user can derive the consideration that products with a lower rice polishing ratio contain more sugars.

As shown on the screen 20, the total content of the four types of sugars in Product A is higher than in Product B. On the other hand, in Product B, the proportion of the three types of oligosaccharides (Disaccharide, Maltotriose, Maltotetraose) in the total sugars is larger than in Product A. Oligosaccharides tend to be less sweet than monosaccharides. Therefore, the user can derive the consideration that Product B provides a mild sweetness and also reduces stimuli such as alcohol and acids to provide a mellow mouthfeel.

The screen 20 is an example of flavor information and is an example of information for taste output using the values of taste-related substances.

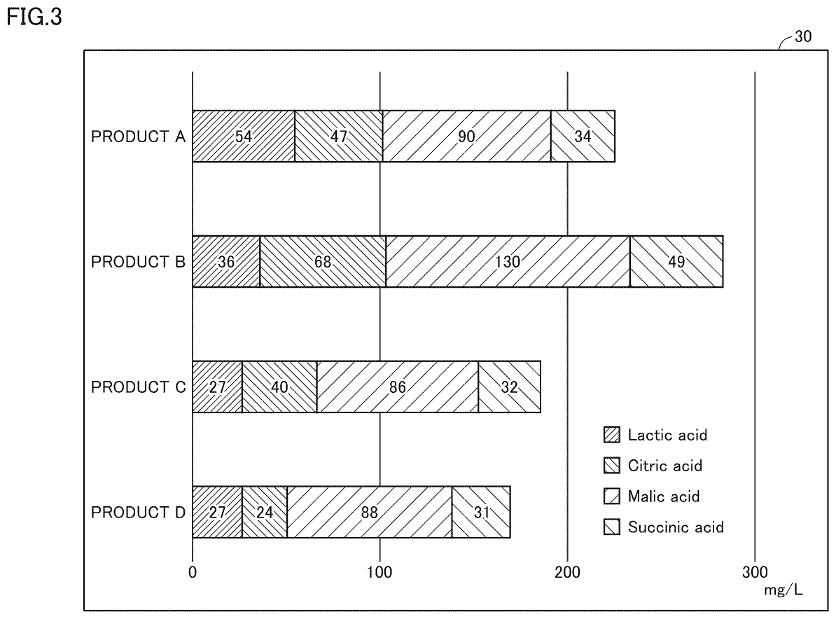

### (Screen 30 of FIG. 3)

The screen 30 of FIG. 3 displays the content of four types of organic acids (Lactic acid, Citric acid, Malic acid, Succinic acid) for each of four types of sake (Products A, B, C, D).

A user can use the information shown on the screen 30 as support for considering the differences in flavor among Products A, B, C, and D.

More specifically, it is said that in sake, organic acids are involved in freshness and a rich, full-bodied, deep taste, also known as “koku”. It is said that the mutual cancellation of sourness and sweetness gives sake a rich taste through their appropriate harmony. In sake, the higher the rice polishing ratio, the relatively higher the content of organic acids. Organic acids contribute to the “koku” of sake. Products C and D (Ginjo-shu, Daiginjo-shu), which showed a tendency to have more sugars, have a relatively low organic acid content as shown in FIG. 3. For this reason, it is considered that Products C and D are perceived as sweeter compared to Products A and B.

Also, the quality of sourness differs depending on the type of organic acid. Malic acid and citric acid have a sourness accompanied by a refreshing sensation, and succinic acid also has a unique umami taste. On the screen 30, a tendency for a high content of malic acid is seen for all of Products A to D. Therefore, based on the results shown on the screen 30, the user can derive the consideration that all of Products A to D give a clean and refreshing impression.

Furthermore, when the ratio of the content of malic acid to the content of succinic acid is 0.8 or more, it is said that sake has a light aftertaste and a sharp finish. On the screen 30, the ratio of malic acid to succinic acid is 0.8 or more for all of Products A to D. Therefore, based on the results shown on the screen 30, the user can derive the consideration that all of Products A to D have a light aftertaste and a sharp finish.

Furthermore, in Product B, the total value of organic acids including succinic acid is larger than in the other products. From this, based on the results shown on the screen 30, the consideration can be derived that Product B has the characteristic of having particular umami and “koku”.

The screen 30 is an example of flavor information and is an example of information for taste output using the values of taste-related substances.

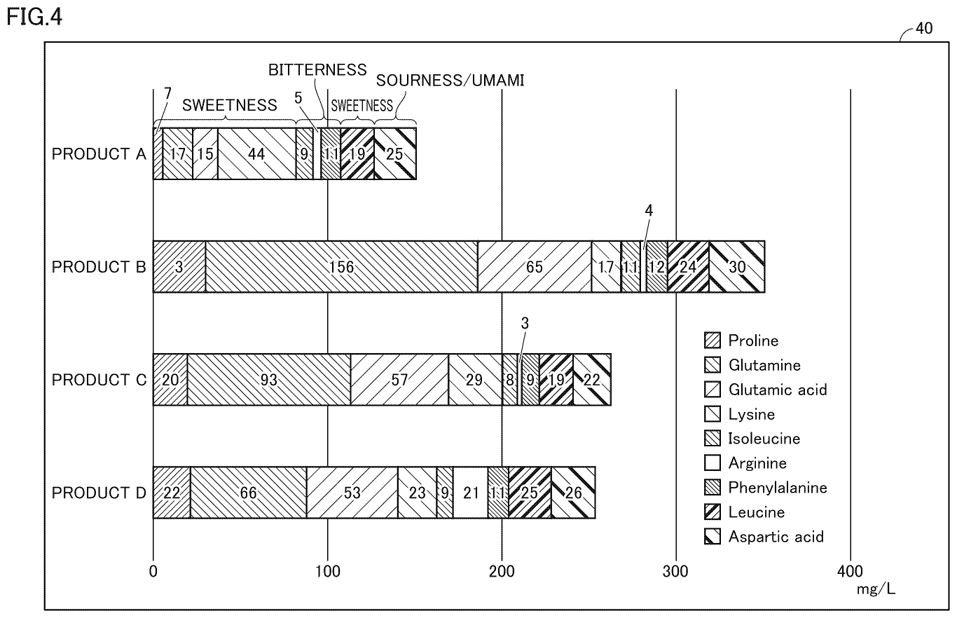

### (Screen 40 of FIG. 4)

The screen 40 of FIG. 4 displays the content of amino acids for each of four types of sake (Products A, B, C, D). The screen 40 shows the content of the following nine compounds.
Proline
Glutamine
Glutamic acid
Lysine
Isoleucine
Arginine
Phenylalanine
Leucine
Aspartic acid

On the screen 40, strings representing the type of taste presented by each compound (sweetness, bitterness, sourness/umami) are appended.

A user can use the information shown on the screen 40 as support for considering the differences in flavor among Products A, B, C, and D.

For example, the total content of amino acids in Product B significantly exceeds that of the other products. It is said that if the amount of amino acids is large, sake exhibits a rich and umami taste, while if it is small, it exhibits a dry taste. Therefore, the user can derive the consideration that Product B exhibits a rich and umami taste, and the remaining products exhibit a dry taste.

Furthermore, Products B to D contain more bitter amino acids (leucine, isoleucine, phenylalanine, and arginine) than Product A. Bitter amino acids give sake sharpness and a spicy (dry) taste. Therefore, the consideration can be derived that Products B to D provide a flavor that gives more sharpness and a spicier (dry) taste compared to Product A.

The screen 40 is an example of flavor information and is an example of information for taste output using the values of taste-related substances.

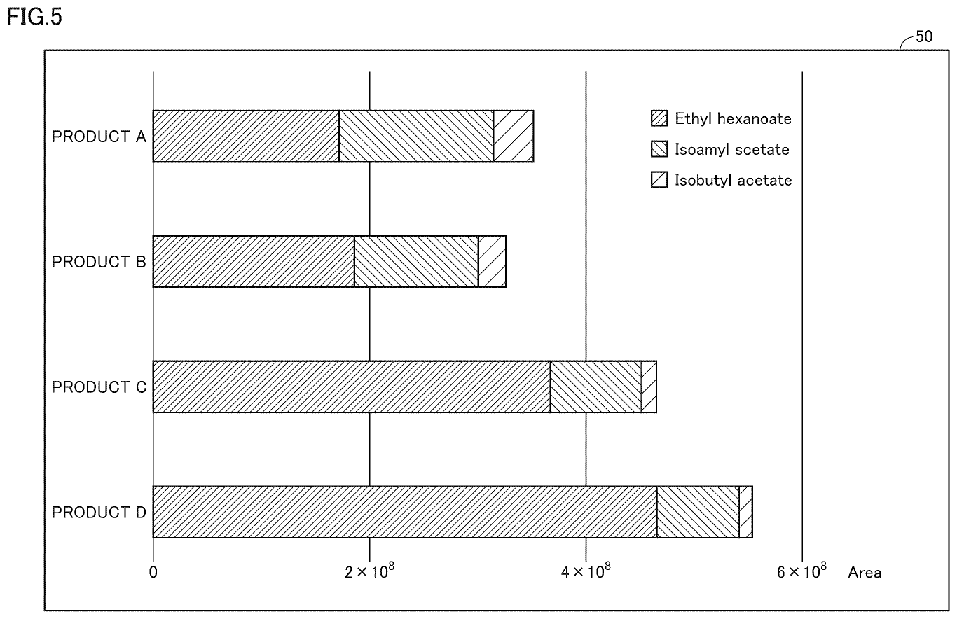

### (Screen 50 of FIG. 5)

The screen 50 of FIG. 5 displays the content of three types of aroma components (Ethyl hexanoate, Isoamyl acetate, Isobutyl acetate) for each of four types of sake (Products A, B, C, D). In one implementation, an aroma component means a volatile substance contained in a food product that has an aroma.

A user can use the information shown on the screen 50 as support for considering the differences in flavor (aroma) among Products A, B, C, and D.

More specifically, the aroma of ethyl hexanoate, which gives a refreshing sensation like green apples or pears, and the aroma of isoamyl acetate and isobutyl acetate, like bananas or melons, are called “ginjo-ka” and are components actually contained in fruits. In Products C and D, ethyl hexanoate accounts for the majority of the aroma components. Therefore, from the screen 50, the user can derive the consideration that Products C and D give a gorgeous impression.

Also, in Products A and B, the ratio of isoamyl acetate and/or isobutyl acetate to ethyl hexanoate is higher than in Products C and D. Therefore, from the screen 50, the user can derive the consideration that Products A and B exhibit an elegantly sweet fruity aroma.

The screen 50 is an example of flavor information and is an example of information for aroma output using the values of aroma-related substances.

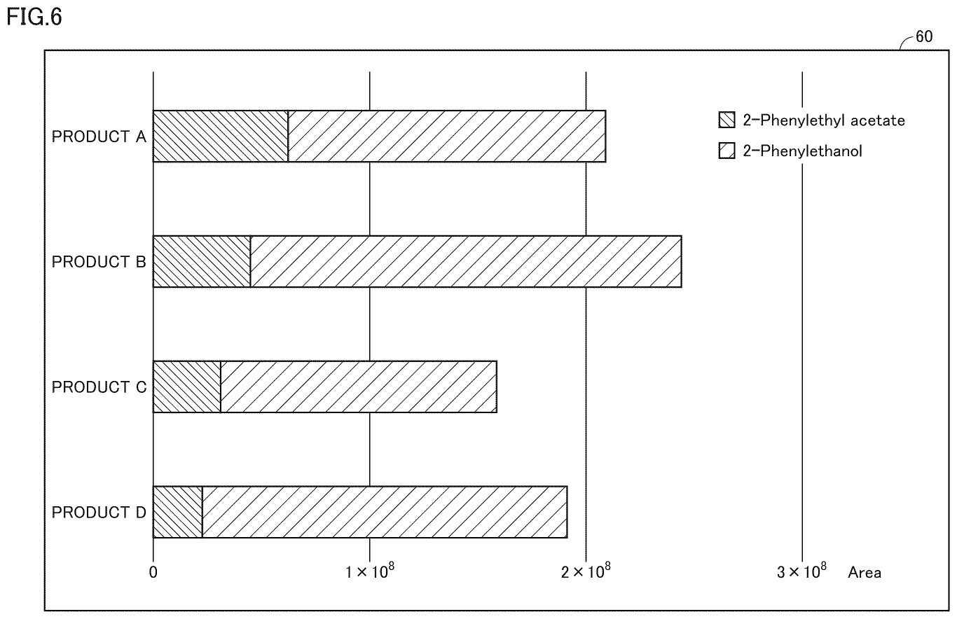

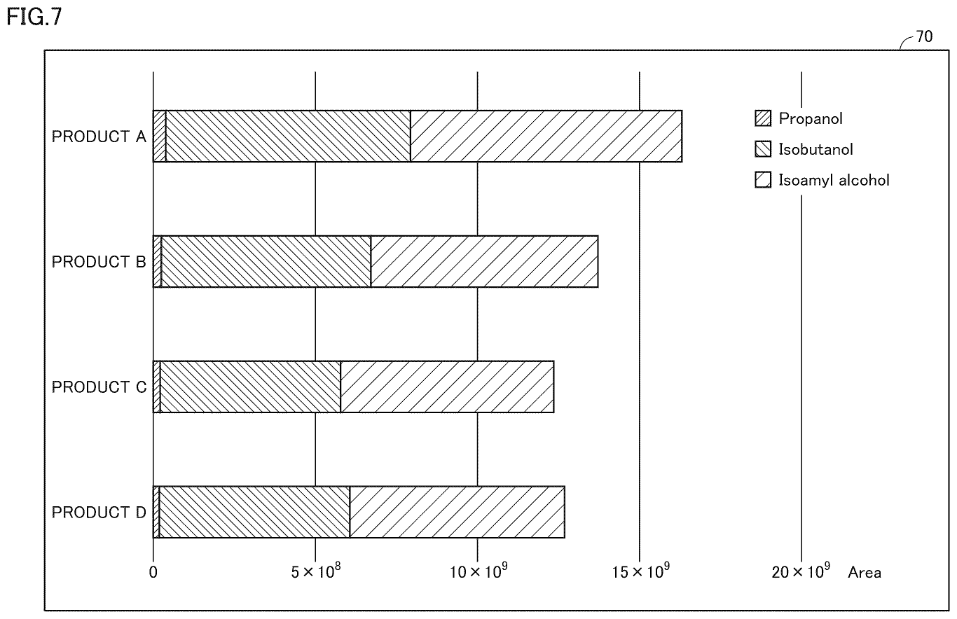

### (Screen 60 of FIG. 6 and Screen 70 of FIG. 7)

The screen 60 of FIG. 6 displays the content of base notes among the aroma components for each of the four types of sake (Products A, B, C, D). More specifically, the screen 60 displays the content of two types of base notes: 2-phenylethanol and 2-phenylethyl acetate.

The screen 70 of FIG. 7 displays the content of three types of fusel oils (Isoamyl alcohol, Isobutanol, Propanol) for each of the four types of sake (Products A, B, C, D).

A user can use the information shown on each of the screens 60 and 70 as support for considering the differences in flavor (aroma) among Products A, B, C, and D.

More specifically, 2-phenylethyl acetate and 2-phenylethanol, which give a rose-like aroma, are components with relatively high boiling points that cause a person to perceive an aroma after taking the sake into their mouth. Such a characteristic is referred to as a base note (fukumi-ka). The base note is the origin of the sake-like aroma in sake.

As shown on the screen 60, Products A and B have a higher content of base notes than Products C and D. From this, the user can derive from the screen 60 the consideration that Products A and B exhibit a sake-like aroma.

Also, as shown on the screen 70, Products A and B have a higher content of fusel oils than Products C and D. From this, the user can derive from the screen 70 the consideration that Products A and B exhibit a mild aroma.

Each of the screens 60 and 70 is an example of flavor information and is an example of information for aroma output using the values of aroma-related substances.

### 3. Flavor Evaluation

The information processing apparatus 100 can output, as an evaluation result for each product, an indicator related to the flavor of each product, calculated using one or more analysis results of each product.

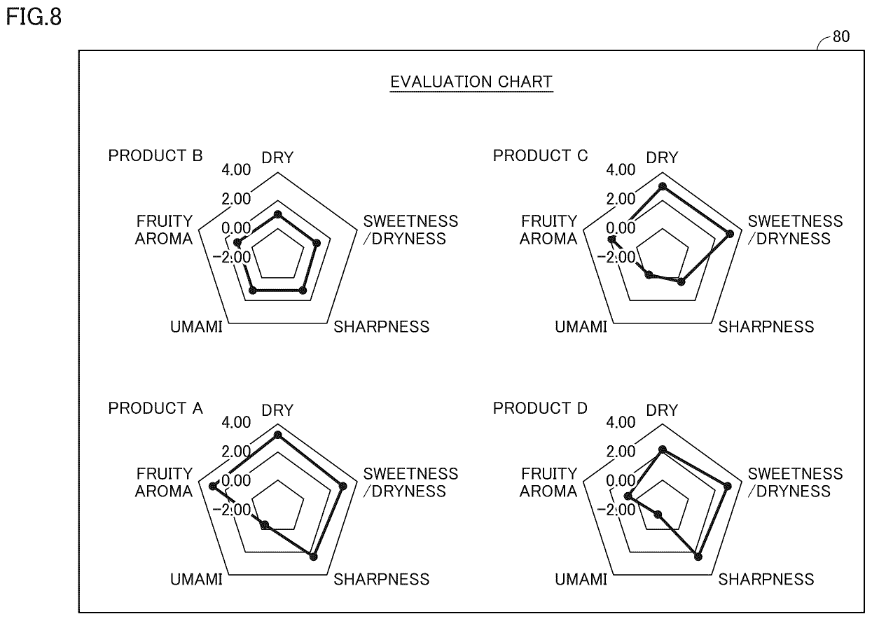

FIG. 8 is a diagram showing an example of a screen output as an evaluation result for four types of products (Products A-D). The screen 80 of FIG. 8 includes a radar chart showing the values of five types of indicators (Dry, Sweetness/Dryness, Sharpness, Umami, Fruity Aroma) for each of Products A-D.

On the screen 80, the radar chart for Product B is shown in the upper left, for Product C in the upper right, for Product A in the lower left, and for Product D in the lower right. On the screen 80, the value of each indicator is normalized so that the value of Product B becomes 1. That is, the indicator value of each product displayed on the screen 80 is normalized based on the indicator value of Product B. The product used as the basis for normalization is also referred to as a “reference product” in this specification. On the screen 80, information identifying the reference product (Product B) may also be displayed.

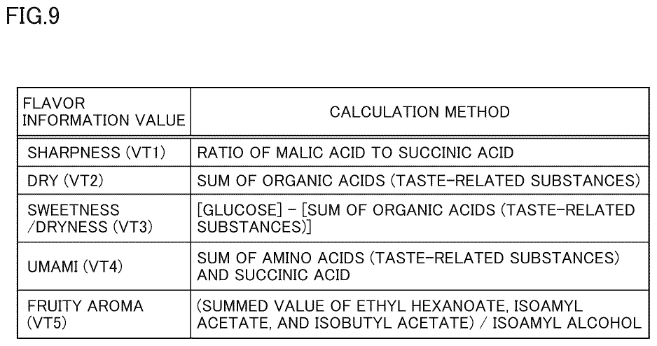

FIG. 9 is a diagram showing an example of a calculation method for the five types of indicators. As shown in FIG. 9, the value Vtl of the indicator “Sharpness” is calculated as the ratio of the content of malic acid to the content of succinic acid in each product.

The value Vt2 of the indicator “dry” is calculated as the sum of the contents of substances identified as “taste-related substances,” which will be described later, among the organic acids in each product. The lower the content of organic acids, the dryer and drier the sake is said to be. From this, it is considered that the smaller the value Vt2 of a certain sake, the higher the degree to which that sake is dry.

Conventionally, the content of all organic acids was sometimes used as an indicator of flavor. On the other hand, in the present embodiment, some organic acids are identified as taste-related substances that affect the difference in flavor among Products A-D. That is, only the content of some organic acids is used as the content of organic acids that affect the difference in flavor among Products A-D. Thereby, the value Vt2 of the indicator “From this, it is considered that the smaller the value Vt2 of a certain sake, the higher the degree to which that sake is dry” is calculated as a value that more accurately represents the difference among Products A-D.

The value Vt3 of the indicator “Sweetness/Dryness” is calculated by subtracting the content of substances identified as “taste-related substances,” which will be described later, among the organic acids from the content of glucose in each product. As described above, in sake, the taste is appropriately harmonized by the mutual cancellation of sourness and sweetness. From this, it is considered that the larger the value Vt3 of a certain sake, the more sweetness it has, and the smaller the value, the more dryness it has.

Similar to the calculation of the value Vt2, the content of substances identified as taste-related substances among the organic acids is also used for the calculation of the value Vt3. Thereby, the value Vt3, like the value Vt2, is calculated as a value that more accurately represents the difference among Products A-D.

The value Vt4 of the indicator “Umami” is calculated as the sum of the content of substances identified as “taste-related substances,” which will be described later, among the amino acids and the content of succinic acid in each product.

Conventionally, the content of all amino acids was sometimes used as an indicator of flavor. On the other hand, in the present embodiment, some amino acids are identified as taste-related substances that affect the difference in flavor among Products A-D. That is, only the content of some amino acids is used as the content of amino acids that affect the difference in flavor among Products A-D. Thereby, the value Vt4 of the indicator “Umami” is calculated as a value that more accurately represents the difference among Products A-D.

The value Vt5 of the indicator “Fruity Aroma” is calculated as the value obtained by dividing the total content of three types of aroma components (ethyl hexanoate, isoamyl acetate, and isobutyl acetate) by the content of isoamyl alcohol in each product. Ethyl hexanoate, isoamyl acetate, and isobutyl acetate are identified as aroma-related substances among the aroma components, as will be described later.

Conventionally, the value obtained by dividing the content of ethyl hexanoate by the content of isoamyl alcohol was sometimes used as an indicator of fruity aroma. On the other hand, in the present embodiment, in addition to ethyl hexanoate, isoamyl acetate and isobutyl acetate can be identified as aroma-related substances that affect the difference in flavor among Products A-D. That is, the contents of more types of substances than conventionally are used as the content of aroma components that affect the difference in flavor among Products A-D. Thereby, the value Vt5 of the indicator “Fruity Aroma” is calculated as a value that more accurately represents the difference among Products A-D.

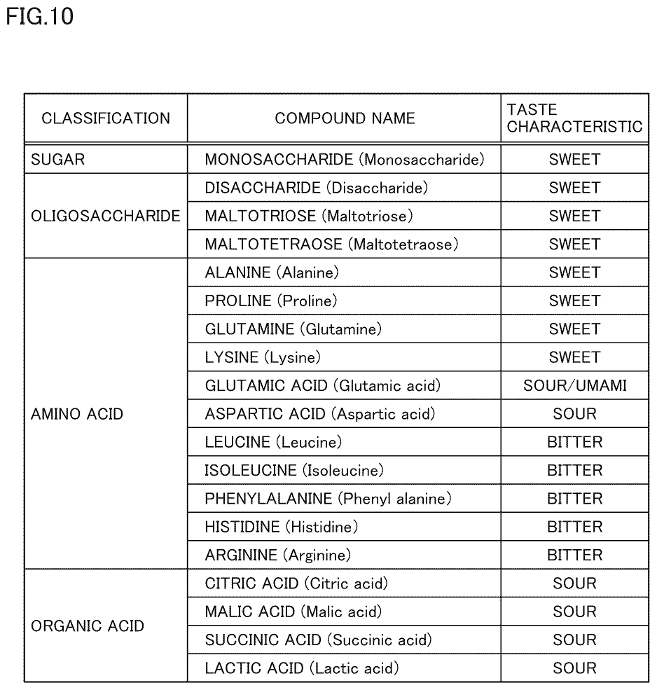

FIG. 10 is a diagram showing an example of compounds identified as taste-related substances. In FIG. 10, the compounds are shown along with their classification and taste characteristics. In one implementation, when the information processing apparatus 100 identifies a taste-related substance, it may acquire the taste characteristic of each taste-related substance by searching a database that associates substances with taste characteristics, generate a table as shown in FIG. 10, and store the table in the storage 102.

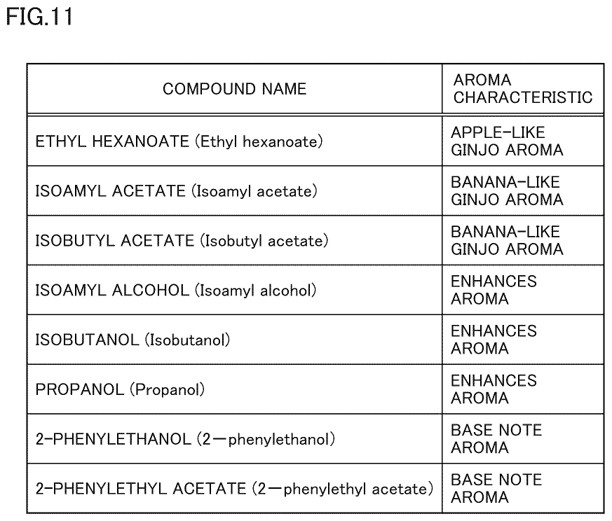

FIG. 11 is a diagram showing an example of compounds identified as aroma-related substances. In FIG. 11, the compounds are shown along with their classification and aroma characteristics. In one implementation, when the information processing apparatus 100 identifies an aroma-related substance, it may acquire the aroma characteristic of each aroma-related substance by searching a database that associates substances with aroma characteristics, generate a table as shown in FIG. 11, and store the table in the storage 102.

The calculation method for each of the five types of indicators is not limited to that shown in FIG. 9. The user may appropriately change the calculation method for the five types of indicators by referring to the types and/or taste or aroma characteristics of the substances identified as taste-related substances and aroma-related substances. Also, the number of indicators to be calculated is not limited to five. It may be one type, or any number of two or more types.

### 4. Identification of Taste-Related Substance

The identification of the taste-related substance will be described.

The information processing apparatus 100 acquires an analysis result of taste components for each of Products A-D. A taste component, also called a taste-imparting component, means a component that provides some kind of taste.

The analysis result represents the respective content of two or more components. For example, when a certain amount of each product is introduced into a liquid chromatograph-mass spectrometer (analysis apparatus 200), the content of each component is derived as the ratio of the peak area of each component to the peak area of an internal standard substance. The information processing apparatus 100 acquires the content of each component from the analysis apparatus 200 as an analysis result of the taste components.

For example, if the contents of 151 types of hydrophilic metabolites (sugars, amino acids, organic acids, nucleosides, nucleotides, etc.) are acquired as the analysis result of the taste components of each product, the analysis results of the four types of products include 604 measurement values (contents).

The information processing apparatus 100 performs an ANOVA (analysis of variance) test on the analysis results of the four types of products. Then, the information processing apparatus 100 identifies, as a taste-related substance, a component corresponding to a measurement value having a p-value of 0.05 or more in the result of this test. An example of the taste-related substances to be identified is the compounds listed in FIG. 10. Thereby, among the plurality of components constituting the flavor, components that particularly affect the difference in flavor can be identified. By identifying substances that significantly affect flavor, it is possible to support a quantitative evaluation of flavor.

The information processing apparatus 100 adopts the ANOVA test as the test for the analysis results of three or more types of products, and adopts the Student's t-test or the Mann-Whitney U test as the test for the analysis results of two types of products.

The information processing apparatus 100 may acquire the analysis results used for identifying the taste-related substance from a gas chromatograph-mass spectrometer.

The components of the taste-related substance to be identified may be limited to only substances included in a component group predetermined for each flavor. For example, for the flavor “From this, it is considered that the smaller the value Vt2 of a certain sake, the higher the degree to which that sake is dry,” components other than organic acids may not be used as taste-related substances. Thereby, components that have a small contribution to the flavor can be removed from the basis of the flavor information.

### 5. Identification of Aroma-Related Substance

The identification of the aroma-related substance will be described.

The information processing apparatus 100 acquires an analysis result of aroma components for each of Products A-D. The analysis result represents the respective content of two or more components. For example, when a certain amount of each product is introduced into a gas chromatograph-mass spectrometer (analysis apparatus 200), the content of each component is derived as the ratio of the peak area of each component to the peak area of an internal standard substance. The information processing apparatus 100 acquires the content of each component from the analysis apparatus 200 as an analysis result of the aroma components.

The information processing apparatus 100 performs an ANOVA (analysis of variance) test on the analysis results of the four types of products. Then, the information processing apparatus 100 identifies, as an aroma-related substance, a component corresponding to a measurement value having a p-value of 0.05 or more in the result of this test. An example of the aroma-related substances to be identified is the compounds listed in FIG. 11. Thereby, among the plurality of components constituting the flavor, components that particularly affect the difference in flavor can be identified. By identifying substances that significantly affect flavor, it is possible to support a quantitative evaluation of flavor.

The information processing apparatus 100 adopts the ANOVA test as the test for the analysis results of three or more types of products, and adopts the Student's t-test or the Mann-Whitney U test as the test for the analysis results of two types of products.

The information processing apparatus 100 may acquire the analysis results used for identifying the aroma-related substance from a liquid chromatograph-mass spectrometer.

The components of the aroma-related substance to be identified may be limited to only substances included in a component group predetermined for each flavor. For example, for the flavor “From this, it is considered that the smaller the value Vt2 of a certain sake, the higher the degree to which that sake is dry” components other than organic acids may not be used as aroma-related substances. Thereby, components that have a small contribution to the flavor can be removed from the basis of the flavor information.

### 6. Flavor Information Value

The information processing apparatus 100 calculates the value of an indicator as shown in FIG. 9 as the flavor information value of each product. That is, the respective values of the indicators Vt1 to Vt5 constitute an example of a flavor information value.

### 7. Processing Flow

### (Main Routine)

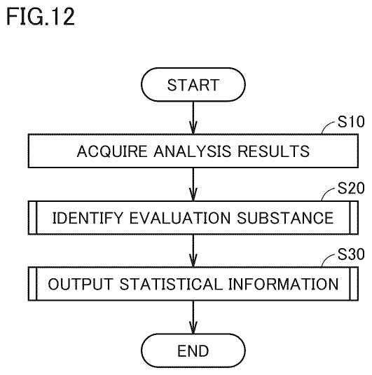

FIG. 12 is a flowchart of a main routine performed in the information processing apparatus 100. In one implementation, the information processing apparatus 100 starts the processing of FIG. 12 in response to an instruction to start being input by a user. In one implementation, the processing described in this specification is realized by one or more processors constituting the CPU 101 executing a given program in the information processing apparatus 100.

In step S10, the information processing apparatus 100 acquires analysis results of two or more food products from the analysis apparatus 200.

In step S20, the information processing apparatus 100 identifies an evaluation substance. The evaluation substance is a general term for the aforementioned taste-related substance and aroma-related substance.

In step S30, the information processing apparatus 100 outputs statistical information of the evaluation substance from among the analysis results acquired in step S10, and then ends the processing of FIG. 12.

### (Identify Evaluation Substance)

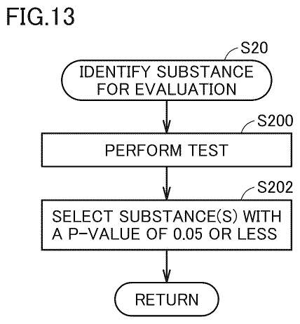

FIG. 13 is a flowchart of the subroutine for step S20 of FIG. 12.

In step S20, in step S200, the information processing apparatus 100 performs a test (the aforementioned ANOVA or Student's t-test or Mann-Whitney U test) on the analysis results acquired in step S10. In step S200, the information processing apparatus 100 may perform a test for each of the taste components and the aroma components. That is, the information processing apparatus 100 may perform a test on the analysis results of the taste components, and also perform a test on the analysis results of the aroma components.

In step S202, the information processing apparatus 100 identifies a substance based on the p-value that is the result of the test in step S200. More specifically, the information processing apparatus 100 identifies a substance having a p-value of 0.05 or less in the result of the test as a taste-related substance or an aroma-related substance. Thereafter, the information processing apparatus 100 returns control to FIG. 12.

### (Output Statistical Information—Output of Flavor Information)

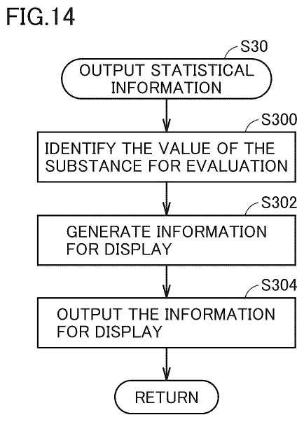

FIG. 14 is a flowchart of a subroutine for step S30 of FIG. 12, performed for outputting flavor information (FIGS. 2-7). In one implementation, an instruction to perform the output of flavor information is input to the information processing apparatus 100. In response to the input of the instruction, the information processing apparatus 100 performs the subroutine of FIG. 14.

In step S30, in step S300, the information processing apparatus 100 identifies the value of the evaluation substance. More specifically, the information processing apparatus 100 extracts the respective analysis result of one or more evaluation substances identified in step S20 from the analysis results acquired in step S10.

In step S302, the information processing apparatus 100 generates screen information (display information) to be displayed as flavor information.

In step S304, the information processing apparatus 100 outputs the display information generated in step S302 to the display device 500. Thereby, a screen described with reference to FIG. 2, etc., is displayed as flavor information. Thereafter, the information processing apparatus 100 returns control to FIG. 12.

Note that the output to the display device 500 is one aspect of the output of flavor information. The flavor information may be output in a mode other than display (for example, audio).

According to the embodiment described above, for each of two or more food products, the respective information value of one or more flavors is output in a state normalized based on one food product. Thereby, information related to the flavor of two or more food products is provided as relative information, and thereby, an accurate evaluation of the two or more food products is provided.

### DESCRIPTION OF REFERENCE NUMERALS

1 Flavor information providing system, 20, 30, 40, 50, 60, 70, 80, 81 Screen, 81A Message, 100 Information processing apparatus, 101 CPU, 102 Storage, 103 Input/output port, 200 Analysis apparatus, 300 Mouse, 400 Keyboard, 500 Display device.

1. A method for providing flavor information related to two or more food products, the method comprising:
acquiring analysis results of respective components of the two or more food products;
identifying, using a result of a statistical test that uses the analysis results of the respective components of the two or more food products, a substance that affects a difference in flavor between the two or more food products; and
outputting, using the analysis result of the identified substance from among the analysis results of the respective components of the two or more food products, flavor information related to the two or more food products.

2. The method according to claim 1, wherein identifying the substance includes identifying a component predetermined for the flavor.

3. The method according to claim 1, wherein
the components include a taste component and an aroma component,
identifying the substance includes:
identifying a taste-related substance from among the taste components; and
identifying an aroma-related substance from among the aroma components, and
outputting the flavor information includes:
outputting information for taste using a value of the taste-related substance; and
outputting information for aroma using a value of the aroma-related substance.

4. The method according to claim 3, wherein both the analysis result of the taste component and the analysis result of the aroma component are measured using a liquid chromatograph or a gas chromatograph.

5. The method according to claim 3, wherein the analysis result of the taste component is measured using a liquid chromatograph, and the analysis result of the aroma component is measured using a gas chromatograph.

6. The method according to claim 1, wherein
the two or more food products are two or more sakes, and
identifying the substance includes identifying one or more amino acids as the substance.

7. The method according to claim 1, wherein
the two or more food products are two or more sakes, and
identifying the substance includes identifying one or more aroma components as the substance.

8. The method according to claim 1, wherein outputting the flavor information related to the two or more food products includes displaying a value of the identified substance together with a type of flavor corresponding to the substance.

9. An information processing apparatus comprising:
one or more processors; and
a storage device storing a program that, when executed by the one or more processors, causes the one or more processors to perform the method according to claim 1.

10. A flavor information providing system comprising:
the information processing apparatus according to claim 9; and
an analysis apparatus that outputs an analysis result of a food product to the information processing apparatus.

11. A non-transitory computer readable medium storing a program that, when executed by one or more processors, causes the one or more processors to perform the method according to claim 1.
